(19) United States
(12) Patent Application Publication
(10) Pub. No.: US 2025/0340806 A1
(43) Pub. Date: Nov. 6, 2025

(54) COMPACT MANNER OF GROWING MICROALGAE SUITABLE FOR CARBON SEQUESTRATION AND CREATING A CIRCULAR ECONOMY

(71) Applicant: ALGAE INTERNATIONAL BHD, Seremban (MY)

(72) Inventor: Jagjit Singh KAURAH, Port Dickson (MY)

(73) Assignee: ALGAE INTERNATIONAL BHD, Seremban (MY)

(21) Appl. No.: 18/871,108

(22) PCT Filed: May 29, 2023
(86) PCT No.: PCT/MY2023/050040

§ 371 Date: Dec. 2, 2024

(30) Foreign Application Priority Data
May 31, 2022 (MY) PI2022002801

(51) Int. Cl. C12M 1/00; C12M 1/12; C12M 1/34; C12M 3/00

(52) U.S. Cl. CPC C12M 21/02; C12M 23/04; C12M 23/22; C12M 23/44; C12M 31/10; C12M 41/06; C12M 41/12

(57) ABSTRACT

There are closed systems of growing microalgae but as they are not able to produce algal biomass economically they are very few and far between and are only used to grow microalgae for very expensive produce. These closed systems also require large space. The result of the lack of economic closed systems is why almost all algal biomass production currently uses raceway ponds. This invention integrates a new compact. very economic and highly productive closed system of growing microalgae including good parameter control with carbon sequestration from say a coal fired power plant or organic waste management in a way that makes the whole process financially attractive and cyclic and if desired it could be made carbon negative. The compact nature of this invention allows algal farms to be sited next to most sources of carbon dioxide.

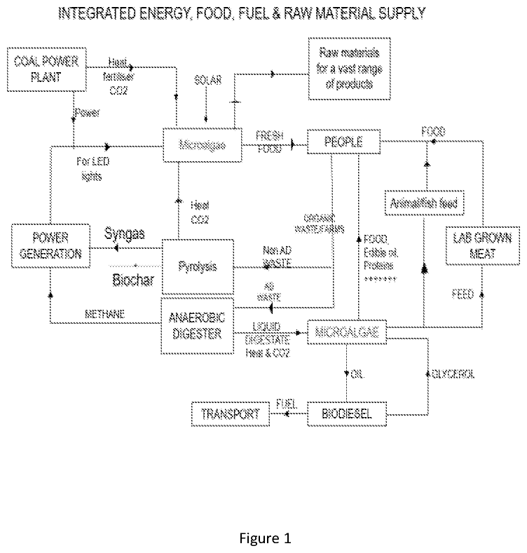

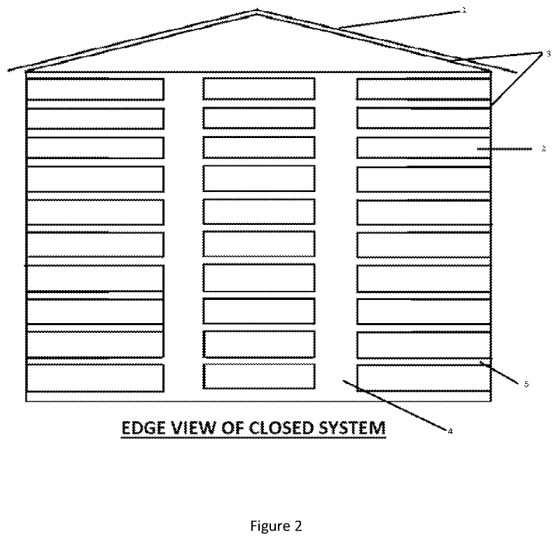

### TECHNICAL FIELD

It is increasingly recognised that carbon sequestration will be needed on a massive scale to keep global average temperature rise to 1.5 degrees centigrade.

The bulk of carbon dioxide (CO2) emitted is by the energy used—73.2% of total global emitted—please see pie chart attached as Drawing 3. Livestock manure, organic waste and industry are also large emitters.

There have been many attempts to sequester carbon but as the costs are unaffordable, there is no general movement to do so.

Even in smaller and simpler possibilities the move to stop climate change is very hesitant. One example is organic waste.

Organic waste is a major problem for the environment despite there being well developed methods of its destruction by various methods including:
Using it for animal feed
Composting
Anaerobic digestion
Pyrolysis
Rendering
Rapid thermophilic digestion
Immobilized enzyme reaction

However, the bulk of organic waste still goes to land fill or is treated in other less attractive ways as the financial rewards for better organic waste management are poor. One example is palm oil mill effluent (POME). Anaerobic digestion is a well-established method of improving the environmental credentials of POME but only a small percentage of mills employ anaerobic digestion as the economic returns are not so attractive. In addition, anaerobic digestion of POME is not a complete solution still requiring unsatisfactory ponding. This invention/innovation adds on improvements to the current processes of handling organic waste while making these processes much more financially attractive. These new inventions/innovations greatly reduce the greenhouse gas effects and could make them carbon negative.

The new innovations involve using microalgae to use up all the by-products of pyrolysis or anaerobic and thermophilic digestion to create zero or near zero waste and producing valuable products to make both processes financially attractive.

In a similar way, this invention/innovation can transform carbon sequestration for the large energy industry

The key to this new process of carbon sequestration and creating a circular industry is a new manner of growing microalgae that is compact with high productivity under tightly controlled conditions.

### BACKGROUND ART

Until the disclosure of this invention, there have been no workable methods of carbon sequestration which are affordable. That is the reason why this urgently needed task is not being undertaken other than on a tiny demo scale by energy suppliers, industry or Governments.

Until the disclosure of this invention there has been no known closed system of commercially growing microalgae which reproduces the conditions of the lab into large scale microalgae culture.

In the much easier to achieve potential of handling animal manure and other organic waste, the progress is patchy at best.

There are several methods of dealing with organic waste including:
Land fill which still handles the largest fraction of organic waste. This is environmentally destructive as methane is given off. Methane is many times more potent as a greenhouse gas than carbon dioxide. If calculated over an 80 year period it's about 80 times more potent.
In addition to the methods listed in item 2 “Technical Field” above there are some minor other methods.
The best of the current methods of handling organic waste like pyrolysis and anaerobic digestion still leave problems like mentioned below.
In the case of pyrolysis carbon dioxide (CO2) and heat are released to the atmosphere and the ash is a waste product.
In the case of anaerobic digestion the digestate discharge still contain organics and equally importantly inorganics. Generally the digestate discharge is treated by ponding like in the case of the POME.

There are many academic papers on how to improve upon the current systems of handling organic waste. However, academic papers unless they can be translated into practical and economic methods of application have no effect on actual practice. There are no known unsubsidised projects commercially and viably using microalgae to create a circular economy and mitigating problems of organic waste profitably.

This invention discloses a set of systems which make the handling of organic waste into a circular economy and in a manner which makes the whole process economically attractive.

This invention also discloses a manner of growing microalgae which enables commercial projects to complete the cycle to a circular economy.

### BRIEF DESCRIPTION OF THE DRAWINGS

Drawing 1 shows how some of the possible processes of energy production and organic waste management link to growing microalgae and how the processes can become cyclic.

Drawing 2 shows the edge view of one iteration of the structure of the module in which microalgae are grown. The module is essentially sealed with walls and roof connected. Herein the connected walls and roof are called the “skin” of the module.

Drawing 3 is a pie chart of the contributions of CO2 by various sectors and activities.

### DISCLOSURE OF INVENTION

This invention integrates a new compact and highly productive system of growing microalgae including good parameter control with carbon sequestration from say a coal fired power plant or handling organic waste management in a way that makes the whole process cyclic and if desired it could be made carbon negative.

Please see Drawing 1 for some possible combinations of processes of the system.

The coal power plant produces electricity which would be used for LEDs to grow microalgae at night, CO2 which would enhance the CO2 in the ambient air supplied to the microalgae to increase their growth rate and heat which would be used to dry the algal biomass. The fertiliser from the power plant ash can be dissolved, cleaned of unwanted components like heavy metals and used to grow microalgae.

The anaerobic digestion depicted in Drawing 1 produces biogas which is used to produce electricity, CO2 and heat, digested solids which can be used as organic fertilizer and digestate liquids which can be used as fertilizer for microalgae. Alternatively, the pyrolysis depicted in Drawing 1 produces syngas which can be used to produce electricity, CO2, heat, biochar which can be used for soil improvement and ash from which fertilizer can be dissolved to grow microalgae. The advantages of these systems being cyclic are that:
The process leaves zero or almost zero waste;
The Carbon Dioxide (CO2) emitted is used to enhance the concentration of CO2 in the air to speed up the growth of microalgae. This could lead to the whole process being carbon negative;
The heat of the flue gas is used to dry the harvested microalgae;
The digestate liquid or the ash provide the fertilizer to grow the microalgae;
The biochar is used for soil improvement and the solid digestate as organic fertilizer for agriculture;
The electricity can be sold or used to continue growing microalgae at night.

The total system produces valuable products like carbon capture and a variety of algal biomass.

### Specific Example and Description of the Drawings

The specific example chosen is mitigation of the problems of chicken droppings and using all products and by-products thereof to grow microalgae. In this case the chicken droppings would be dried and pyrolysed. The overall process is in line with the process shown in Drawing 1.

The pyrolysis gives off syngas which could be used to produce electricity in a gas engine. The gas engine would emit CO2 as part of the hot exhaust gas typically at about 350 to 400 degrees centigrade. In this system the hot exhaust gas would be channelled to transfer the heat to clean air through a heat exchanger. The heated clean air dries the microalgae usually using a spray drying system. The flue gas exiting the heat exchanger would typically be between 70 to 100 degrees centigrade. This would then be cleaned up of anything detrimental to the growth of microalgae using any of the known systems. The cleaned CO2 rich flue gas would be used to enrich ambient air of its CO2 content up to the level suitable for the particular microalgae species being grown. The microalgae use up both the CO2 from the flue gas and the ambient air. The CO2 enhanced air supply temperature can be adjusted to mitigate temperature change in the module growing the microalgae. For example, the temperature could be lower during the day and higher during the night.

For optimal growth of microalgae, the daily temperature variation of the culture must be limited to 4 degrees centigrade. This is one of the major reasons why growth rate in labs is higher than in the farm. There are several ways to reduce temperature variation in the module like make the skin such that it is efficient at reducing outside temperature changing the inside temperature, switching on more LEDs at night than during the day and adjusting the temperature of the CO2 enriched air supplied to the microalgae culture can have its temperature adjusted to even out temperature variation within the module as shown in drawing 2

From the pyrolysis vessel biochar and ash would be removed and the biochar sold to farmers for soil improvement. Biochar is remarkably effective in improving the productivity of agricultural soils. The fertiliser in the ash would be dissolved and used to grow the microalgae.

The electricity produced would be used for operations and to power LED's to continue growing microalgae during the night.

Drawing 2 shows the edge view of one iteration of the structure of the module in which microalgae are grown.

Item 1 are a set of solar panels above the clear sealed skin of the module.

Item 2 is one of the trays containing the culture in which microalgae grow. The number of trays in a module varies on the design best suited to the location, land size and other requirements. One possible arrangement is 10 trays high, as shown in Drawing 2, one above another with 3 rows of them side by side. Length wise it could be any number and one option is to have 3 sets lengthwise too. Hence, in this case there would be 3×3×10 transparent trays. It is best not to cover the transparent trays for easy operation. The trays are best made of transparent material for maximum light reaching the microalgae culture from all directions.

Item 3 is the outer transparent skin of the module. The material for this can be chosen from a range transparent plastics suitable for outdoor use. It is important that this outer skin is as air tight as possible so that combined with positive air pressure inside the module, outside air cannot come into the module.

Item 4 is the working area between the trays. This has to comply with local requirements.

Item 5 is the gap between the trays. The gap height can be set at what is comfortable and practical.

The conditions of temperature and temperature variation can be controlled by covering the module with solar panels and air supply temperature varied to stabilise daily temperature variation like cooler air supply during the day and warmer air supply during the night, lighting levels during the day can be controlled both by the percentage shade provided and by the number of LEDs lighted up and during the night by the number of LEDs lighted up. This allows ideal growth conditions, similar to lab conditions, for microalgae to be maintained 24 hours per day.

In most locations of power plants, other large CO2 emitters and where organic waste is at least anaerobically digested or pyrolysed there is limited space. This invention of stacked or vertical farming of microalgae requires much less space than other systems.

### INDUSTRIAL APPLICABILITY

The key areas of industrial applicability are:
Carbon sequestration made viable;
Food security;
Creating a cleaner environment by introducing cyclic industries;
The very compact nature of the microalgae growing system allows locations with limited land to benefit;
Path to zero waste and carbon negative possibilities.

1. A manner of growing microalgae in a closed system module wherein the module comprises:
i) multiple levels of transparent trays (2) in which the microalgae grow in its culture;
ii) the microalgae grow in transparent growth trays (2) that are stacked one above another with a gap (5) between them; and
iii) The module has a transparent skin (3) which allows the conditions inside the module to be controlled;
and wherein the conditions within the closed system module are controlled to optimise growth conditions for microalgae including temperature and lighting conditions.

2. (canceled)

3. The manner of growing microalgae as claimed in claim 1, wherein the transparent trays are stacked vertically in a multi-level manner to hold the culture growing microalgae.

4. The manner of growing microalgae as claimed in claim 1, wherein the closed system module is covered by solar panels.

5. The manner of growing microalgae as claimed in claim 1, wherein the daily temperature change is moderated by means of switching on more LED lights at night.

6. The manner of growing microalgae as claimed in claim 1, wherein the daily temperature change is moderated by means of adjusting the temperature of the CO2 enriched air supplied to the culture.

7. A comprehensive manner of handling organic waste wherein most of the products of such handling being carbon dioxide, heat, digestate solids, digestate liquids, biochar, ash and electricity are used to produce useful products by growing microalgae.

8. The manner of handling organic waste as claimed in claim 5, wherein the waste heat generated is used to dry the harvested algal biomass.

9. The manner of handling organic waste as in claim 5, wherein the waste carbon dioxide is used to grow microalgae.

10. The manner of handling organic waste as in claim 5, wherein the waste digestate liquid is used as a fertiliser to grow microalgae.

11. The manner of handling organic waste as in claim 5, wherein the waste ash is used as a source of fertiliser to grow microalgae.

12. The manner of handling organic waste as in claim 5, wherein the waste carbon dioxide is used to enrich carbon dioxide from the atmosphere to grow microalgae where the entire process could become net carbon negative.
